(19) United States
(12) Patent Application Publication
(10) Pub. No.: US 2012/0116289 A1
(43) Pub. Date: May 10, 2012

(54) SHOCKWAVE VALVULOPLASTY DEVICE WITH GUIDEWIRE AND DEBRIS BASKET

(76) Inventors: DANIEL HAWKINS, BELLEVUE, WA (US); JOHN M. ADAMS, SNOHOMISH, WA (US); TOM GOFF, MOUNTAIN VIEW, CA (US)

(21) Appl. No.: 13/232,730

(22) Filed: Sep. 14, 2011

Related U.S. Application Data
(60) Provisional application No. 61/411,798, filed on Nov. 9, 2010.

(51) Int. Cl. A61B 17/20; A61F 2/01

(52) U.S. Cl. 604/22; 606/200

(57) ABSTRACT

A valvuloplasty system comprises a balloon adapted to be placed adjacent leaflets of a valve. The balloon is inflatable with a liquid. The system further includes a shock wave generator within the balloon that produces shock waves. The shock waves propagate through the liquid and impinge upon the valve to decalcify and open the valve. The balloon is carried on a catheter that includes a guidewire lumen. The system further includes a debris collecting basket carried on the catheter proximal to the balloon.

### PRIORITY CLAIM

The present application claims the benefit of copending U.S. Provisional Patent Application No. 61/411,798, filed Nov. 9, 2010, which application is incorporated herein by reference in its entirety.

### BACKGROUND OF THE INVENTION

Aortic calcification, also called aortic sclerosis, is a buildup of calcium deposits on the aortic valve in the heart. This often results in a heart murmur, which can easily be heard with a stethoscope over the heart. However, aortic calcification usually doesn't significantly affect the function of the aortic valve.

In some cases, though, the calcium deposits thicken and cause narrowing at the opening of the aortic valve. This impairs blood flow through the valve, causing chest pain or a heart attack. Doctors refer to such narrowing as aortic stenosis.

Aortic calcification typically affects older adults. But when it occurs in younger adults, it's often associated with an aortic valve defect that is present at birth (congenital) or with other illnesses such as kidney failure. An ultrasound of the heart (echocardiogram) can determine the severity of aortic calcification and also check for other possible causes of a heart murmur.

At present there is no specific treatment for aortic calcification. General treatment includes the monitoring for further developments of heart disease. Cholesterol levels are also checked to determine the need for medications to lower cholesterol in the hope to prevent progression of aortic calcification. If the valve becomes severely narrowed, aortic valve replacement surgery may be necessary.

The aortic valve area can be opened or enlarged with a balloon catheter (balloon valvuloplasty) which is introduced in much the same way as in cardiac catheterization. With balloon valvuloplasty, the aortic valve area typically increases slightly. Patients with critical aortic stenosis can therefore experience temporary improvement with this procedure.

Unfortunately, most of these valves narrow over a six to 18 month period. Therefore, balloon valvuloplasty is useful as a short-term measure to temporarily relieve symptoms in patients who are not candidates for aortic valve replacement. Patients who require urgent noncardiac surgery, such as a hip replacement, may benefit from aortic valvuloplasty prior to surgery. Valvuloplasty improves heart function and the chances of surviving non-cardiac surgery. Aortic valvuloplasty can also be useful as a bridge to aortic valve replacement in the elderly patient with poorly functioning ventricular muscle. Balloon valvuloplasty may temporarily improve ventricular muscle function, and thus improve surgical survival. Those who respond to valvuloplasty with improvement in ventricular function can be expected to benefit even more from aortic valve replacement. Aortic valvuloplasty in these high risk elderly patients has a similar mortality (5%) and serious complication rate (5%) as aortic valve replacement in surgical candidates.

The present invention provides an alternative treatment system for stenotic or calcified aortic valves. As will be seen subsequently, the embodiments described herein provide a more tolerable treatment for aortic stenosis and calcified aortic valves than the currently performed aortic valve replacement. The invention also provides a more effective treatment than current valvuloplasty therapy. For patients undergoing trans aortic or catheter based aortic valve replacement the invention can soften, smooth, and open the aortic valve annulus more effectively than current valvuloplasty and prepare the area for the catheter delivered valve.

Current valvuloplasty therapy can dislodge calcium particles which may flow down stream and cause blockage in smaller arteries. Such blockage can even occur in larger arteries, such as the carotid artery, for example. Carotid artery blockage is especially worrisome because the carotid arteries provide blood to the brain. Any blockage in a carotid artery could result in stroke or even death.

### SUMMARY OF THE INVENTION

The invention provides a valvuloplasty system comprising a balloon adapted to be placed adjacent leaflets of a valve. The balloon is inflatable with a liquid. The invention further comprises an embolic protection basket and a shock wave generator within the balloon that produces shock waves that propagate through the liquid for impinging upon the valve. The embolic protection basket is arranged for collecting debris resulting from the shock waves impinging upon the valve.

The embolic protection basket is self-deployable. The system may further comprise an elongated tube. The balloon may be carried by the elongated tube and the embolic protection basket may also be carried on the elongated tube. The balloon may be at a distal end of the elongate tube and the embolic protection basket may be carried on the elongated tube proximal to the balloon. The embolic protection basket may include a tubular extension extending proximally from the embolic protection basket. The embolic protection basket and the tubular extension may be carried on the elongated tube.

The shock wave generator may include a first electrical arc generator and a second electrical arc generator. The electrical arc generators may comprise at least one electrode adapted for connection to a voltage pulse generator. Each of the electrical arc generators may comprise an electrode pair adapted for connection to a voltage pulse generator.

The system may further comprise an elongated tube having a lumen. The balloon may be carried by and about the elongated tube. The system may further include a guidewire adapted to slidingly receive the lumen of the elongated tube.

The embolic protection basket may be carried on the elongated tube. The balloon and the embolic protection basket may be arranged on the elongated tube such that when the balloon is within the leaflets of an aortic valve, the embolic protection basket is distal to a brachiocephalic trunk. The system may further comprise an elongated over tube arranged to be received over the elongated tube, the balloon, and the embolic protection basket. The embolic protection basket and the balloon may be arranged to be in a collapsed state while being in the over tube. The system embolic protection basket may be further arranged to expand into a deployed state when exiting the over tube.

The invention further provides a valvuloplasty system comprising an elongated tube having a proximal end, a distal end, and a longitudinal lumen extending there through and a balloon carried on the distal end of the elongated tube and adapted to be placed adjacent leaflets of a valve. The balloon is inflatable with a liquid. The system further comprises a guidewire slidingly received by the longitudinal lumen of the elongated tube for guiding the elongated tube and the balloon along a desired path and a shock wave generator within the balloon that produces shock waves that propagate through the liquid for impinging upon the valve.

The system may further comprise an embolic protection basket carried on the elongated tube proximal to the balloon. The shock wave generator comprises an electrical arc generator. The electrical arc generator may comprise at least one electrode adapted for connection to a voltage pulse generator.

The invention still further provides a valvuloplasty method of treating a valve having leaflets and an annulus. The method comprises the steps of placing a balloon adjacent to the leaflets of the valve, placing an embolic protection basket proximal to the balloon, and inflating the balloon with a liquid. The method further includes the steps of producing shockwaves within the balloon that propagate through the liquid for impinging upon the valve leaflets and the valve annulus, and capturing debris resulting from the shockwaves impinging upon the valve annulus and leaflets within the embolic protection basket.

The placing step may be performed by placing the balloon on opposite sides of the valve leaflets. Alternatively, the placing step is performed by placing the balloon within the valve annulus.

The invention still further provides a valvuloplasty method for treating a valve having leaflets and an annulus comprising the steps of providing an elongated tube having a proximal end, a distal end, and a longitudinal lumen extending there through, a balloon, inflatable with a liquid, carried on the distal end of the elongated tube and adapted to be placed adjacent leaflets of a valve, a guide wire, and a shock wave generator within the balloon that produces shock waves that propagate through the liquid for impinging upon the valve and advancing the guide wire along a desired path through the valve annulus. The method further includes the steps of sliding the elongated tube onto the guide wire, advancing the elongated tube on the guide wire until the balloon is adjacent the valve leaflets, inflating the balloon with the liquid, and producing shockwaves within the balloon with the shock wave generator.

The method may further comprise the further steps of providing the elongated tube with an embolic protection basket proximal to the balloon, and capturing debris resulting from the shockwaves impinging upon the valve annulus and leaflets within the embolic protection basket.

### BRIEF DESCRIPTION OF THE DRAWINGS

The features of the present invention which are believed to be novel are set forth with particularity in the appended claims. The various described embodiments of the invention, together with representative features and advantages thereof, may best be understood by making reference to the following description taken in conjunction with the accompanying drawings, in the several figures of which like reference numerals identify identical elements, and wherein:

FIG. 1 is a cut away view of the left ventricle, the aorta, and the aortic valve of a heart showing a reduced aortic valve open area and thickened valve leaflets due to calcium and fibrotic tissue;

FIG. 2 is a cut away view of the aortic valve of a heart with a treatment balloon placed on both sides of the aortic valve leaflets, according aspects of the present invention;

FIG. 3 is a schematic view of a valvuloplasty system employing a dual shockwave balloon according to aspects of the invention;

FIG. 4 is a cut away view of a heart showing an alternate valvuloplasty shock wave balloon according to a further aspects of the present invention;

FIG. 5 is cut away view of a valvuloplasty system including a shockwave balloon, deployed in relation to aortic valve leaflets, having a center guide lumen sized to be received over a guide wire and a guide tube which is used to deliver the shockwave balloon to the desired area of the heart according to further aspects of the invention;

FIG. 6 is a cut away view of a heart showing the balloon of FIG. 5 in position about the aortic valve leaflets and carried on the guide wire; and

FIG. 7 is a partial cut away view of a heart to an enlarged scale showing an aorta with a balloon of a valvuloplasty system according to the invention positioned within the aortic valve and an embolic protection basket deployed with respect to the balloon to capture debris resulting from a valvuloplasty procedure performed with the valvuloplasty system.

### DETAILED DESCRIPTION OF THE INVENTION

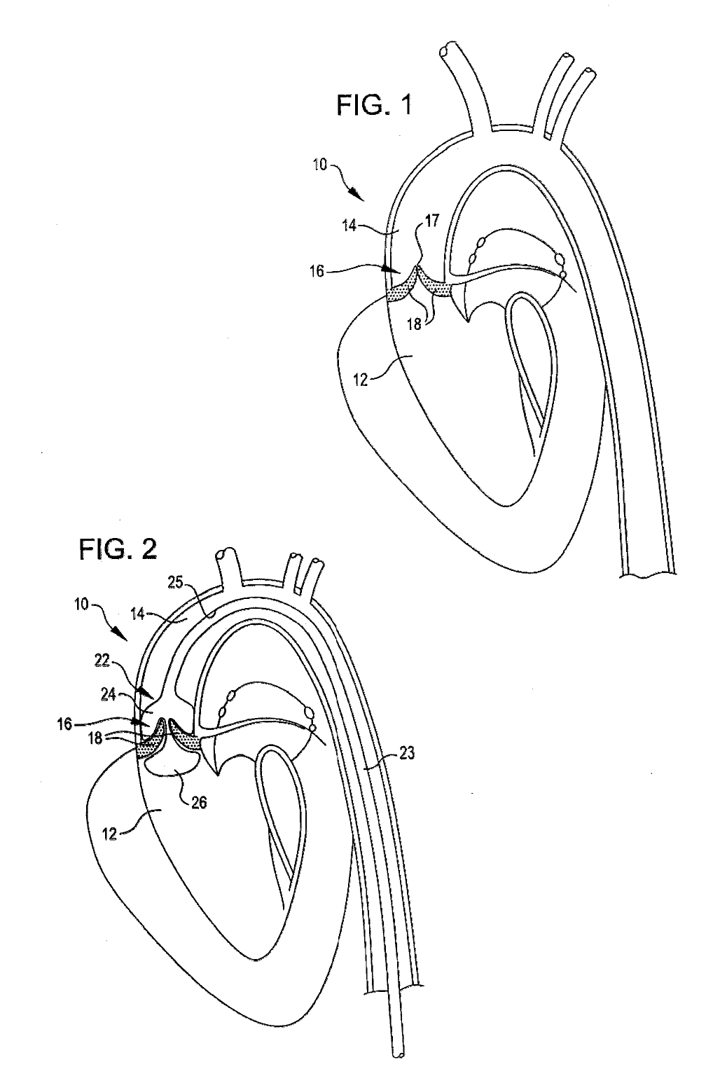

Referring now to FIG. 1, it is a cut away view of the left ventricle 12, the aorta 14, and the aortic valve 16 of a heart 10 with a stenotic and calcified aortic valve 16. Here more particularly, it may be seen that the opening 17 of the stenotic and calcified aortic valve 16 is restricted in size and that the valve leaflets 18 are thickened with calcium deposits and fibrotic tissue. The thickened leaflets 18 and smaller valve opening 17 restrict blood flow from the heart creating excess work for the heart 10 and poor cardiac output. As previously mentioned, current treatment includes replacement of the valve or attempts to stretch the valve annulus with a balloon.

FIG. 2 is a cut away view of the aortic valve 16 with a treatment balloon 22 placed on both sides of the aortic valve leaflets 18. The balloon 22 may be formed from a compliant or a non-compliant material. The balloon, as seen in FIG. 2, is at the distal end of an elongated tube 23. The treatment balloon 22 has two longitudinally spaced chambers 24 and 26 that share a common inflation lumen 25 of the tube 23. Alternatively the balloon chambers 24 and 26 may not share the same inflation fluid path. The chambers 24 and 26 are longitudinally spaced such that chamber 24 is positioned on one side of the aortic valve leaflets 18 and chamber 26 is positioned on the other side of the aortic valve leaflets 18. The chambers 24 and 26 are inflated with saline/contrast mixture, for example. Each chamber 24 and 26 may contain an electrode (as shall be seen subsequently) that can produce electrical arcs to deliver timed shock waves. The shock waves can be synchronized to concurrently impinge upon both sides of the leaflets 18 to maximize the effectiveness of breaking calcium deposits. Such shock waves may be generated and also synchronized to the R wave of the heart 10 in a manner as described for example in co-pending application No. 61/061,170 filed on Jun. 13, 2008, which application is incorporated herein in its entirety.

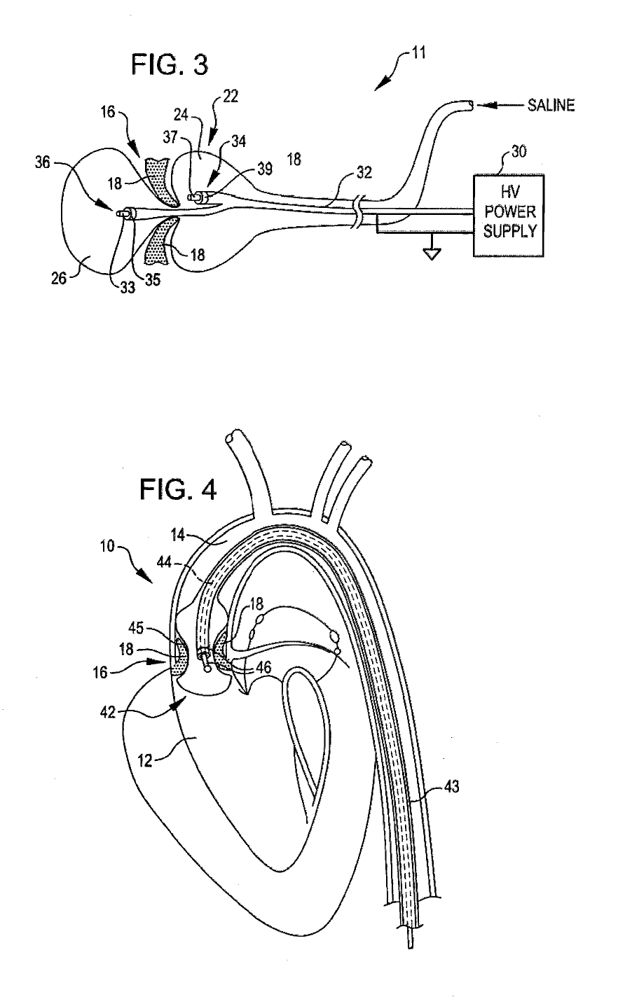

FIG. 3 is a schematic view of a valvuloplasty system 11 that includes the dual shockwave balloon 22. The balloon 22 has received a high voltage catheter 32 that is connected to a high voltage power supply 30. The schematic representation shows the positioning of the balloon chambers 24 and 26 above and below the leaflets 18 of the aortic valve 16. As previously described, shock waves will impinge upon opposite sides of the leaflets 18 to more effectively break calcium deposits in the valve leaflets 18. The annulus will also be treated in this arrangement. To that end, the high voltage catheter 32 includes electrode pairs 34 and 36 that are coaxially arranged electrodes placed in chambers 24 and 26 respectively of the balloon 22. More specifically, electrode pair 34 is at the distal end of a first cable and comprises a center conductor 33 and an outer conductive shield 35. Similarly, electrode pair 34 is at the distal end of a second cable and comprises a center conductor 37 and an outer conductive shield 39. High voltage pulses from power supply 30 are applied to the electrode pairs 34 and 36 in a manner as described in the aforementioned application Ser. No. 61/061,170 to create shockwaves within the fluid within the chambers 24 and 26 of the balloon 22. The shock waves impinge upon the valve leaflets 18 and the valve annulus to cause the break up of calcium deposits and fibrotic tissue on the valve leaflets 18 and annulus to open and smooth the aortic valve 16.

FIG. 4 shows an alternate valvuloplasty shock wave balloon 42 at the distal end of an elongated tube 43. The balloon 42 is placed in the annulus of the aortic valve 16. To that end, the balloon 42 has a reduced diameter portion 45 for being received within the valve annulus. The balloon 42 has a high voltage catheter 44 therein that terminates in an electrode pair 46. As in the previous embodiment, the electrode pair 46 may comprise a pair of coaxially arranged electrodes where a center conductor may form at least a part of one electrode and at an outer conductive shield may form at least a part of the other electrode. The catheter 44 and its electrode pair 46 provide shock waves as previously described. Such an arrangement will decalcify the leaflets 18. This not only will decalcify the leaflets 18, but will also soften the aortic valve annulus and expand its diameter. Hence, the balloon 42 provides the added advantage of exerting expansion pressure directly to the annulus of the valve to remodel the annulus diameter.

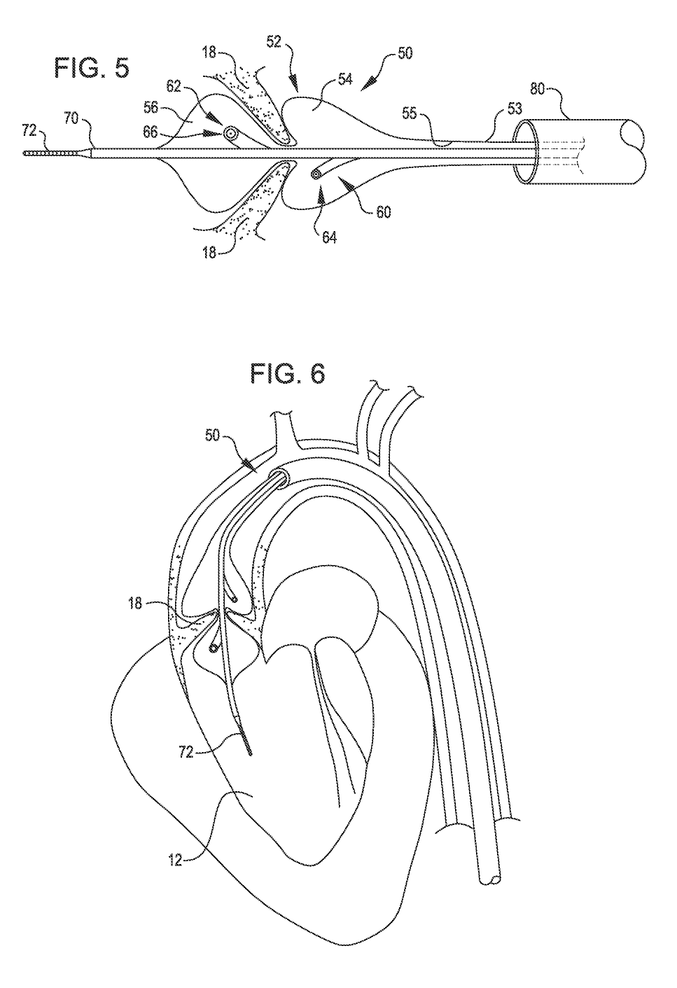

FIG. 5 is a cut away view of a valvuloplasty system 50 embodying the present invention including a shockwave balloon 52 deployed on both sides of the aortic valve leaflets 18. The balloon 52 may be formed from a compliant or a non-compliant material. The balloon, as seen in FIG. 5, is at the distal end of an elongated tube 53. The treatment balloon 52 has two longitudinally spaced chambers 54 and 56 that share a common inflation lumen 55 of the tube 53. Alternatively the balloon chambers 54 and 56 may not share the same inflation fluid path. The chambers 54 and 56 are longitudinally spaced such that chamber 54 is positioned on one side of the aortic valve leaflets 18 and chamber 56 is positioned on the other side of the aortic valve leaflets 18. The chambers 54 and 56 may be inflated with saline/contrast mixture, for example.

The system 50 further includes a shockwave generator including electrical arc generators 60 and 62. Each of the electrical arc generators 60 and 62 includes an electrode pair 64 and 66, respectively. The electrode pairs may include coaxially disposed electrodes similar to the electrodes of electrode pairs 34 and 36 of FIG. 3.

Each balloon chamber 54 and 56 contains one of the electrodes pairs. As seen in FIG. 5, balloon chamber 54 has electrode pair 64 and balloon chamber 56 has electrode pair 66. The elongated tube 53 further includes a center guide lumen 70. The center guide lumen is sized to fit over a guide wire 72. Also, shown is a guide tube 80 which is used to deliver the shockwave balloon to the desired area of the heart.

As in previous embodiments, the balloon chambers 54 and 56 may be expanded with a mixture of saline and contrast which aides in shock formation and visualization via x-ray. An added benefit to contrast is the absorption of UV light waves generated by the arc of the shockwave generators.

The shock waves can be synchronized to concurrently impinge upon both sides of the leaflets 18 to maximize the effectiveness of breaking calcium deposits. Such shock waves may be generated and also synchronized to the R wave of the heart 10 in a manner as described for example in co-pending application No. 61/061,170 filed on Jun. 13, 2008, which application is incorporated herein in its entirety.

FIG. 6 is a cutaway view showing the system 50 of FIG. 5 with the balloon in position about the aortic valve 18 and carried on the guide wire 72. The guide wire 72 is placed through the aortic valve and in the left ventricle 12 to direct the placement of the system 50. For simplicity the electrodes are not shown in FIG. 6.

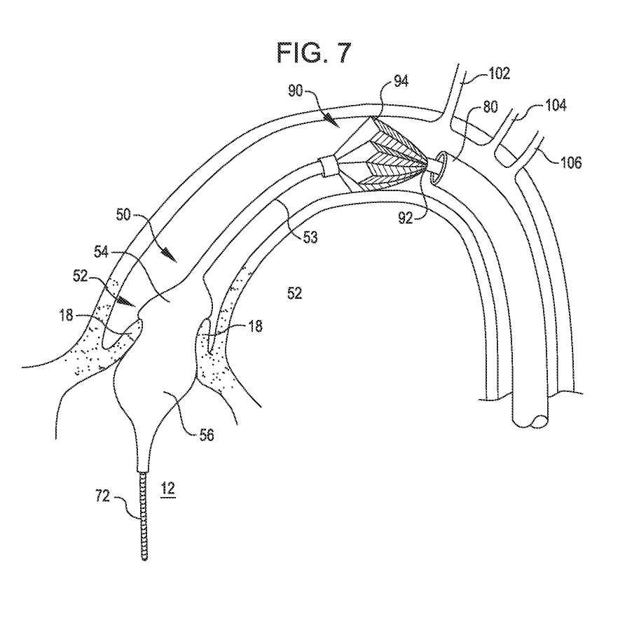

FIG. 7 is a partial cut away view of the aorta and the aortic valve of a heart to an enlarged scale together with a valvuloplasty system embodying the invention. The valvuloplasty system shown in FIG. 7 is the valvuloplasty system 50 of FIG. 5 but further including an embolic protection basket 90 carried on the elongated tube 53 proximal to the balloon 52. The embolic protection basket 90 is also deployed on the elongated tube 53 distal to the brachiocephalic trunk 102, the common carotid artery 104 and the subclavian artery 106. With the embolic protection basket thus positioned, it will capture debris resulting from a valvuloplasty procedure performed with the valvuloplasty system 50 and prevent such debris from entering the brachiocephalic trunk 102, the common carotid artery 104 or the subclavian artery 106.

The embolic protection basket 90 may be fixed to the elongated tube 53. Alternatively, the basket may include a proximal extension 92 to permit the basket 90 to be slidingly disposed on the elongated tube 53. This would allow the relative distance between the basket 90 and the balloon 52 to be adjusted.

The basket preferably has an umbrella-like structure 94 formed of nitinol, for example. As is well known, nitinol has shape memory permitting the basket to be placed into the introduction guide tube 80 in a collapsed state. When the guide tube 80 is pulled back, the basket 90 will be freed and expand from the collapsed state to an expanded and deployed state as shown.

In use of the system 50 and the embolic protection basket 90, the guide wire 72 is first advanced into the heart and through the aortic valve leaflets 18. The distal tip of the guide wire will extend into the left ventricle 12. Next, the guide tube 80 is advance over the guide wire 72 until it is just past the valve leaflets 18. The system 50, together with the embolic protection basket 90 is then guided down the guide tube 80 on the guide wire 72. Once the balloon 52 is adjacent the valve leaflets 18, the guide tube is pulled back to expose the balloon 52. The guide tube is pulled back further until the embolic protection basket 90 is exposed. This frees the basket to expand from the collapsed state to the expanded deployed state. The balloon 52 may now be inflated to form the chambers 54 and 56.

Electrical energy pulses may now be applied to the system 50 to create shock waves in the balloon chambers 54 and 56. As previously described, the shock waves can be synchronized to concurrently impinge upon both sides of the leaflets 18 to maximize the effectiveness of breaking calcium deposits. Such shock waves may be generated and also synchronized to the R wave of the heart in a manner as previously described. During the procedure, the basket captures debris dislodged by the shock waves to protect against such debris from entering the brachiocephalic trunk 102, the common carotid artery 104 or the subclavian artery 106 (and hence the brain) while at the same time allowing blood to flow through those arteries. After use, the system 50 and debris can be retracted into the over tube 80 and removed from the body.

While particular embodiments of the present invention have been shown and described, modifications may be made, and it is therefore intended to cover all such changes and modifications which fall within the true spirit and scope of the invention.

What is claimed is:

1. A valvuloplasty system, comprising:
a balloon adapted to be placed adjacent leaflets of a valve, the balloon being inflatable with a liquid;
an embolic protection basket; and
a shock wave generator within the balloon that produces shock waves that propagate through the liquid for impinging upon the valve, the embolic protection basket being arranged for collecting debris resulting from the shock waves impinging upon the valve.

2. The system of claim 1, wherein the embolic protection basket is self-deployable.

3. The system of claim 1, further comprising an elongated tube, wherein the balloon is carried by the elongated tube, and wherein the embolic protection basket is carried on the elongated tube.

4. The system of claim 3, wherein the balloon is at a distal end of the elongate tube and wherein the embolic protection basket is carried on the elongated tube proximal to the balloon.

5. The system of claim 4, wherein the embolic protection basket includes a tubular extension extending proximally from the embolic protection basket.

6. The system of claim 5, wherein the embolic protection basket and the tubular extension are carried on the elongated tube.

7. The system of claim 1, wherein the shock wave generator comprises a first electrical arc generator and a second electrical arc generator.

8. The system of claim 7, wherein each of the electrical arc generators comprises at least one electrode adapted for connection to a voltage pulse generator.

9. The system of claim 7, wherein each of the electrical arc generators comprises an electrode pair adapted for connection to a voltage pulse generator.

10. The system of claim 1, further comprising an elongated tube having a lumen, wherein the balloon is carried by and about the elongated tube, and wherein the system further includes a guidewire adapted to slidingly receive the lumen of the elongated tube.

11. The system of claim 10, wherein the embolic protection basket is carried on the elongated tube.

12. The system of claim 11, wherein the balloon and the embolic protection basket are arranged on the elongated tube such that when the balloon is within the leaflets of an aortic valve, the embolic protection basket is distal to a brachiocephalic trunk.

13. The system of claim 11, further comprising an elongated over tube arranged to be received over the elongated tube, the balloon, and the embolic protection basket.

14. The system of claim 13, wherein the embolic protection basket and the balloon are arranged to be in a collapsed state while being in the over tube.

15. The system of claim 13, wherein the embolic protection basket is further arranged to expand into a deployed state when exiting the over tube.

16. A valvuloplasty system, comprising:
an elongated tube, the elongated tube having a proximal end, a distal end, and a longitudinal lumen extending there through;
a balloon carried on the distal end of the elongated tube and adapted to be placed adjacent leaflets of a valve, the balloon being inflatable with a liquid;
a guidewire slidingly received by the longitudinal lumen of the elongated tube for guiding the elongated tube and the balloon along a desired path; and
a shock wave generator within the balloon that produces shock waves that propagate through the liquid for impinging upon the valve.

17. The system of claim 16, further comprising an embolic protection basket carried on the elongated tube proximal to the balloon.

18. The system of claim 17, wherein the shock wave generator comprises an electrical arc generator.

19. The system of claim 18, wherein the electrical arc generator comprises at least one electrode adapted for connection to a voltage pulse generator.

20. A valvuloplasty method of treating a valve having leaflets and an annulus, comprising the steps of:
placing a balloon adjacent to the leaflets of the valve;
placing an embolic protection basket proximal to the balloon;
inflating the balloon with a liquid;
producing shockwaves within the balloon that propagate through the liquid for impinging upon the valve leaflets and the valve annulus; and
capturing debris resulting from the shockwaves impinging upon the valve annulus and leaflets within the embolic protection basket.

21. The method of claim 20, wherein the placing step is performed by placing the balloon on opposite sides of the valve leaflets.

22. The method of claim 20, wherein placing step is performed by placing the balloon within the valve annulus.

23. A valvuloplasty method for treating a valve having leaflets and an annulus, comprising:
providing an elongated tube having a proximal end, a distal end, and a longitudinal lumen extending there through, a balloon, inflatable with a liquid, carried on the distal end of the elongated tube and adapted to be placed adjacent leaflets of a valve, a guide wire, and a shock wave generator within the balloon that produces shock waves that propagate through the liquid for impinging upon the valve;
advancing the guide wire along a desired path through the valve annulus;
sliding the elongated tube onto the guide wire and advancing the elongated tube on the guide wire until the balloon is adjacent the valve leaflets;
inflating the balloon with the liquid; and
producing shockwaves within the balloon with the shock wave generator.

24. The method of claim 23, comprising the further steps of providing the elongated tube with an embolic protection basket proximal to the balloon; and capturing debris resulting from the shockwaves impinging upon the valve annulus and leaflets within the embolic protection basket.
